SEQUENCE LISTING (2 sequences)

SEQ ID NO: 1 - DNA, 31 bp, Artificial Sequence
  Description of Artificial Sequence Synthetic primer
tttgaccgtgcagcytaayrcatgcaagtcg

SEQ ID NO: 2 - DNA, 33 bp, Artificial Sequence
  Description of Artificial Sequence Synthetic primer
tttgaccgtgcagyycacgygttackcacccgt

(19) United States
(12) Patent Application Publication
(10) Pub. No.: US 2007/0003924 A1
(43) Pub. Date: Jan. 4, 2007

(54) Serial analysis of ribosomal and other microbial sequence tags

(75) Inventors: Zhongtang Yu, Lewis Center, OH (US); Mark Morrison, Dublin, OH (US); Marie Yu, (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 11/157,072

(22) Filed: Jun. 20, 2005

Related U.S. Application Data
(60) Provisional application No. 60/580,846, filed on Jun. 18, 2004.

(51) Int. Cl. C12Q 1/70; C12Q 1/68; C12P 19/34

(52) U.S. Cl. 435/5; 435/6; 435/91.2

(57) ABSTRACT

A simple and robust method for genetic analysis of complex microbial communities involves the steps of PCR amplification of V1 region of rrs genes in the community DNA sample using two universal primers, followed by cleavage by BsgI and removal of dual-biotinylated primers using streptavidin-coated magnetic beads to purify RSTs, and concatemerization of the RSTs and size selection of resultant concatemers by agarose gel electrophoresis. The isolated concatamers are then cloned and sequenced and subjected to sequence analyses to enable identification of the members of the microbial community.

### PRIORITY CLAIM

This application claims priority to U.S. Provisional Patent Application 60/580,846, filed Jun. 18, 2004, which is incorporated herein by reference, in its entirety.

### GOVERNMENT SUPPORT

This invention was supported, at least in part, by grants 11320-5300501 from the Ohio Board of Regents, 56320-530189 MORRM from the State of Ohio, and OHOG0592-500486 and OHOA1075-550019 from the Agricultural Research and Development Center.

### FIELD OF THE INVENTION

The present invention relates to methods, compositions, and kits for rapid, high throughput analysis of microbial community diversity. The invention is based in part on improvements to techniques developed for serial analysis of ribosomal sequence tags.

### BACKGROUND

Two decades of research using various molecular techniques to evaluate microbial communities has revealed the most complex and concentrated, yet largely uncultivated and unknown, pools of microbial diversity ever examined. It is been estimated that a soil sample as small as one gram may have hundreds to thousands of microbial species, each of which can be represented by up to billions of individuals. This vastness of diversity and complexity in microbial community structure presents a unique challenge to comprehensive characterization of microbial communities. While such comprehensive characterization is integral to understanding the role of microbial communities in those processes that determine ecosystem functions, and those that affect human health, animal nutrition, and the environment, technology constraints significantly limit the feasibility of such comprehensive characterization.

One approach to unveil diversity and community composition in microbial communities is to determine and identify ribosomal RNA (rrs) gene sequences through PCR amplification, cloning, and sequencing of individual rrs genes from bacterial isolates or microbial community samples. However, this approach is not sufficiently cost-effective or efficient to afford comprehensive examination of microbial communities because only one rrs can be sequenced per sequencing reaction. For example, with regard to an ideal microbial community containing 200 bacterial species at an equal abundance of 10⁸ cells/species, an equally large number of random clones must be sequenced in order to identify each species. Of course, such an ideal microbial community does not exist. If one of the 200 bacteria is one log and another two logs less abundant than the rest, then a substantially greater number (probably more than 40,000) of clones must be sequenced in order to determine the rrs of most species in the community. Using current technology, such large-scale sequencing is not feasible for most microbial ecological studies. Thus, even in the most ambitious efforts reported so far, only hundreds of clones were sequenced per clone library, and not a single microbial community has ever been characterized comprehensively after two decades of extensive studies using molecular techniques.

Another approach to increase the efficiency and throughout of sequencing-based methods is to determine multiple sequences per sequencing reaction. Serial analysis of gene expression (SAGE) is an approach that permits identification of multiple mRNA species in eukaryotes per sequencing reaction. SAGE revolutionized the expression sequence tag (EST) analysis that identifies one mRNA per sequencing reaction. A similar strategy is used in the serial analysis of ribosomal sequence tags (SARST) method. SARST employs a series of enzymatic reactions to amplify and ligate (using two linkers) ribosomal sequence tags (RSTS) of the entire V1 region of rrs into concatemers, which are subsequently cloned and sequenced. Consequently, SARST permits the determination of multiple rrs sequences per sequencing reaction. This novel tool offers a substantial increase (up to 20 fold) in throughput over the conventional rrs-cloning-sequencing approach. Despite its advantages over the limited and conventional methods, the SARST procedures are time, material, and labor intensive, requiring the use of linkers, several repeated endonuclease digestions and ligations, and three rounds of purification using magnetic beads and PAGE, hence diminishing its usefulness to achieve rapid, high throughput analysis of microbial communities.

What is lacking in the art is a streamlined and robust method of analysis of the genetic makeup of microbial communities which involves a minimum number of reagents, and can accommodate large numbers of samples for evaluation in a short time frame.

### SUMMARY OF THE INVENTION

Disclosed herein are improved and robust analytical methods for characterizing and profiling the phylogenetic diversity in microbial communities.

The present invention provides a method for the rapid and comprehensive analysis of complex microbial communities. Through a series of enzymatic reactions, microbial DNA isolated from a microbial community is subject to PCR techniques to produce tags that are representative of unique regions in the microbial DNA. The PCR techniques are performed with primers having extensions designed to enable direct concatenation of tags without the need for intermediate linkers. After isolation, the tags are subsequently processed to produce concatemers comprising two or more sequence tags on a single DNA molecule. The concatemers are then cloned and sequenced to identify and quantify the sequence tags.

In some embodiments, the inventive methods described herein involve an improved serial analysis of ribosomal sequence tags (iSARST) comprising the steps of: PCR amplifying a DNA sample, such as of genomic DNA, from a microbial community with primers having complementarity to a targeted region of DNA, wherein the primers have extensions comprising restriction endonuclease recognition sites which, upon digestion with corresponding restriction endonuclease reagents, produces isolated polynucleotides referred to as “tags” comprising overhangs that are complementary at their 3′ and 5′ ends; isolating the tags; digesting the tags with the corresponding restriction endonuclease reagents; separating the tags from the primers; concatenating the tags in a head to tail orientation; purifying, cloning and analyzing the sequences of the concatemers using conventional techniques. In some embodiments, the targeted region of DNA is a variable or hypervariable region, such as, for example, the V1 region of 16S rrs gene. In other embodiments, the targeted region may be some other genetic region of interest. In the various embodiments of this invention, tags from within the targeted genetic regions are amplified and isolated using primer pairs which flank the region of interest. The primer design is based on known or predicted sequence characteristics of such flanking regions, and primers comprise sequences having complementarity to such flanking regions as well as extensions which encode restriction endonuclease recognition sites specifically selected to provide tags having complementary ends or overhangs.

In some embodiments, the methods of the present invention may be used for examination of the V1 region of the 16S rrs gene in a microbial community, which is a region of high variability. According to this embodiment, good results have been obtained according to the process comprising the following steps: PCR amplification of V1 region of 16S rrs gene using two universal primers encoding a BsgI restriction endonuclease site, digestion with the corresponding BsgI restriction endonuclease, separation and purification of RSTs, concatenation of the RSTs forming concatemers, cloning and sequencing of purified RST concatemers, and RST sequence analysis (FIG. 1). The primers used in the PCR step to target the V1 region of 16S rrs gene are universal primers. As appropriate, such primers may comprise additional elements such as, for example, biotin labels, or other such labels which facilitate the subsequent separation of the primers from the tags. In the case where biotin labels are used with primers, separation of the primers from the tags is achieved by passage of the primer/tag sample over streptavidin-coated magnet beads. Of course, other techniques are well known in the art for facilitating separation of primers from their complementary polynucleotides.

Good results have been obtained using the following primer pair: BsgI-Bact64f (5′-dual biotin-TTT GAC CGT GCA GCY TAA YRC ATG CAA GTC G-3′) and BsgI-Bact109r1 (5′-dual biotin-TTT GAC CGT GCA GYY CAC GYG TTA CKC ACC CGT-3′), wherein the corresponding restriction endonuclease is BsgI, and wherein the BsgI-Bact64f differs from the universal primer Bac64f-BpmI with a different extension, longer primer length (18 bases), and reduced degeneracy (8, instead of 16) and wherein the BsgI-Bact109r1 is the same as the bacterial primer 109r1 which is known in the art based on the work of Lane, but with a unique extension. Of course, different primers may be designed to target the V1 region of 16S rrs gene. Likewise, different genetic regions may be targeted using the described method wherein the specific primers are designed to enable specific isolation of tags within such regions.

In alternate embodiments, the methods described herein may be used to isolate and characterize sequence tags based on targeting other genetic sites of interest. For example other hypervariable regions such as, for example, the V2-V9 regions of ribosomal genes can be targeted according to the methods described herein to isolate sequence tags from within those regions. Additionally, different microbial phyla, orders, classes, families, genera, and species can be specifically analyzed using this method by designing appropriate primers to selected targeted regions.

In other alternate embodiments, the methods described herein may be used to isolate and characterize genetic material based on targeting yet other genetic sites of interest, such as genetic regions encoding specific types of genes. For example genes involved in antibiotic and antimicrobial resistance may be targeted using specifically designed primer sets to enable characterization of the resistance profile of microbes in a microbial community.

The invention also provides genetic constructs and polynucleotides encoding specific sequence tags that are produced according to the described methods. The invention also provides kits for evaluating microbial populations, such kits comprising one or more primer sets in appropriate containers each of said one or more primer sets comprising primer pairs for targeting specific genetic regions in the microbial genomes; reaction components in appropriate containers comprising restriction endonucleases corresponding to and specific for the restriction endonuclease recognition sites on the primer set, at least one ligase for producing concatemers, such as T4 ligase, DNA polymerase, T4 DNA polymerase, all in appropriate containers; and a cloning vector in an appropriate container.

According to the methods of the present invention, thorough and comprehensive examination of microbial diversity, community composition, and structure can be accomplished cost-effectively in typical microbiology laboratories. This novel methodology permits the analysis of a large number of DNA sequences in a minimum number of steps, with a reduction in required reagents and time as compared to prior methods. The present methods, compositions and kits are useful to achieve a virtually complete inventory of microorganisms and their relative abundance present in various microbial communities. By correlating this comprehensive information and various biotic and abiotic parameters in the habitat, the methods of the present invention are useful to developing an understanding of the role of these microbial communities in processes that determine ecosystem functions, and processes that affect the environment, and nutrition and health of humans and animals.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying figures, which are incorporated in and constitute a part of this specification, and together with the description, serve to explain the principles of the invention.

### BRIEF DESCRIPTION OF THE DRAWINGS

FIG. 1. shows a schematic representation of iSARST. The V1 region of rrs gene is amplified by PCR using universal primers preceded by an extension containing a BsgI recognition site and a dual biotin label at their 5′ ends. Following digestion with BsgI and purification, the RSTs are concatemerized in a head-to-tail orientation and cloned after blunt-end polishing. The RST concatemers in the clone libraries are sequenced, and the grouped RSTs ( [symbol] Ý95% sequence identity) are annotated using rRNA sequence databases.

FIG. 2. shows the relative abundance (%) of individual RSTs assigned to the nine bacterial phyla identified from the RST library. The value following each phylum name indicates the abundance of individual phyla relative to the total number of RST (of 1,055 RSTs). The numbers in parentheses are derived from 9 different conventional rrs clone libraries prepared from rumen samples and cited in the text. The first number is the percentage (of 457 rrs clones sequenced) assigned to the same phylum, and the second number represents the number of different rrs clone libraries containing such a clone.

FIG. 3. shows the prevalence of RSTs affiliated to genera within Firmicutes. Unclassified genus 1 is within Clostridiaceae; unclassified genus 2 is within Lachnospiraceae; unclassified genus 3 is within Eubacteriaceae; and unclassified genus 4 is within Acidaminococcaceae. V1-RSTs sequence identity varies among different genera. For instance, it ranges from 32.2% to 100% among the type strains of the true Clostridium (RDP II, Release 9.0), while among the type strains of Ruminococcus, it ranges from 31.7% to 95%. Given the sequence ID of all the RSTs is greater than 45% with known sequences, all RSTs were assigned to the closest genera.

FIG. 4. shows the prevalence of RSTs affiliated to genera within Bacteroidetes. Unclassified genus 1 is within Porphyromonadaceae, and unclassified genus 2 is within Saprospiraceae. Again, V1-RSTs sequence identity varies among different genera. It ranges from 50.0% to 98.1% among the type strains of Bacteroides, while among the type strains of Prevotella, it ranges from 44.8% to 98.1%. Similarly as for Firmicutes, all RSTs were assigned to the closest genera.

### DETAILED DESCRIPTION OF THE INVENTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to that this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The disclosure of all patents, patent applications (and any patents that issue thereon, as well as any corresponding published foreign patent applications), GenBank and other accession numbers and associated data, and publications mentioned throughout this description are hereby incorporated by reference herein. It is expressly not admitted, however, that any of the documents incorporated by reference herein teach or disclose the present invention.

Background

As the vast microbial diversity and complexity of microbial communities are more fully examined and appreciated; numerous studies and persistent efforts have been devoted to deciphering such diversity and complexity. However, none of the methodologies feasibly permit thorough and comprehensive characterization of any microbial community. The advent of SARST improved the ability to more comprehensively characterize microbial communities. With respect to throughput capacity, SARST to cloning-sequencing of individual rrs genes is SAGE to EST, as both are based on the same strategy: sequencing multiple sequences per reaction. Despite the advances permitted by SARST, the method is nevertheless laborious and reagent intensive, requiring six oligonucleotides (2 PCR primer sets, and two linkers), of which four are labeled with expensive dual biotins, six different enzymes, two magnetic bead purification steps, and one PAGE purification. The SARST approach is also time intensive, taking from 7-8 working days to construct a SARST library. These disadvantages diminish the usefulness of SARST in studies where a large number of microbial samples must be examined.

By streamlining and simplifying the lengthy and time-consuming SARST procedures, the methods according to the present invention substantially improve upon the large scale efficacy of SARST while retaining its high-throughput capacity (up to 19 rrs genes per sequencing reactions). Within three working days, one researcher with basic training in molecular biology techniques can construct several RST libraries that contain sufficient RSTs representing most, if not all, members of a microbial community. Within 1-2 weeks, a typical microbiology laboratory can determine thousands or more unique RSTs, probably adequate to identify most microbial community members. The methods according to the present invention result in an overall reduction of at least 50% of enzymes, reagents, cost, and procedures as compared to SARST and other techniques. The methods of the current invention are more cost effective than SARST, and can be readily, and affordably, implemented in a kit format to make comprehensive analysis of microbial communities even more convenient and efficient.

Moreover, in addition to enabling significant time, cost, and materials savings, the methods of the present invention also significantly improve the feasibility of completely characterizing any complex microbial community. An example of such a diverse and complex community is the type that resides in gastrointestinal tracts of humans and animals. It is believed that these complex communities play a key role in nutrition and health. Previous studies using known methodologies (PCR-DGGE followed by re-amplification and sequencing of DGGE bands; and cloning and sequencing of individual 16S rrs genes) suggested limited diversity in these gastrointestinal tract microbial communities. Even the most ambitious and comprehensive studies only sequenced several hundreds of clones (and thus rrs genes) per clone library. Using the methods of the present invention, thousands or even hundred thousands of 16S rrs sequences can be determined efficiently. Such high-throughput capacity is highly useful for completely characterizing any complex microbial community, such as the ones present in gastrointestinal tracts and environments such as water treatment and waste processing facilities, and other bioremediation facilities.

Sequence Tag Analysis

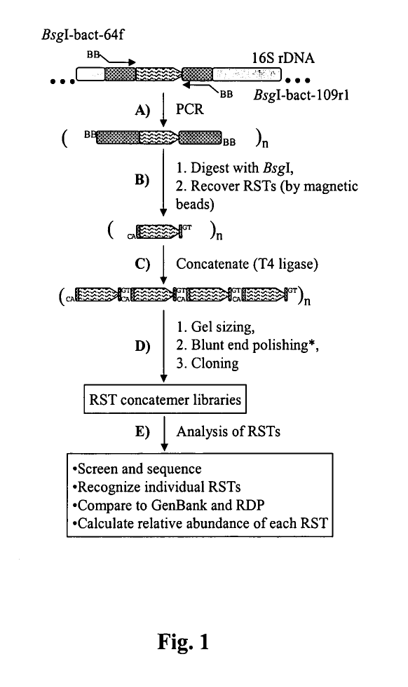

The approach described herein is substantially streamlined and simplified (FIG. 1) compared to prior methods for profiling microbial communities, particularly as compared to SARST. This simplification is achieved through use of primers having extension which enable direct concatenation of tags without the need for intermediate linkers. In one embodiment, primers such as BsgI-Bact64f and BsgI-Bact109r1 may be used, wherein the primers target the V1 hypervariable region of the 16s rrs gene, and comprise extensions comprising restriction endonuclease recognition sites such that upon digestion with corresponding restriction endonuclease reagents the resulting tags comprise overhangs that are complementary at their 3′ and 5′ ends.

Unlike the SAGE method, where sequence tags can be concatenated in head-head or tail-tail orientation without interfering in subsequent PCR screening or DNA sequencing, RSTs are ligated in head-tail orientation in SARST. Otherwise, the concatemers will be difficult to amplify and sequence due to the formation of stem-loop structure by adjacent RSTs of similar sequences. This obstacle is imposed by the nature the rrs sequences. In SARST, head-tail orientation was ensured by the use of linkers and subsequent digestion and tricky RST concatenation in the presence of two endonucleases (SpeI and NheI). Consequently, SARST is a lengthy time-consuming process.

According the invention described herein, primers such as the BsgI-Bact64f and BsgI-Bact109r1 primer pair were designed to provide an amplicon that generates RSTs with compatible 3′ overhangs (5′-GT-3′ for the sense strand and 3′-CA-5′ for the antisense strand) following digestion with BsgI. The resultant RSTs can thus be ligated directly in head-tail orientation to form RSTs concatemers which are ready to be cloned following size selection and end polishing. The inventive methods have eliminated the need for the two biotinylated linkers used in SARST.

By eliminating the need for two linkers, the inventive methods described herein have also eliminated all the steps required by SARST to ligate, digest, and remove the linkers—thus, the following steps have been eliminated: ligation of the two linkers to RSTs, PAGE purification of the linkers-RSTs ligation products, double digestion with SpeI and NheI to release the RSTs again, the second purification of RSTs with magnetic beads, and the awkward RST concatenation in the presence of SpeI and NheI. In comparison to the SARST approach, the methods of the present invention require fewer enzymes, oligos and reagents, and may be performed in less than half the time (7-8 working days for SARST as compared to about 3 working days according to embodiments according to the present invention).

Microbial Samples and Diversity

While the inventive methods described herein have been used with microbial community DNA from the rumen, the methods are equally applicable to other types of samples which contain numerous species of microorganisms, such as gastrointestinal, stool, oral plaque, vaginal, soil, sludge, landfill, bioreactors, wastewater and other aquatic samples. The methods are particularly useful for application to microbial community analysis of human gastrointestinal tract. For instance, the methods described herein can be used to identify the bacteria implicated in inflammatory bowel diseases, which are thought to be caused by multiple yet unknown bacteria. Such application will advance our understanding how this important microbial community impacts human nutrition and health. Application of the present methods and compositions may also assist in tracking the response of microbial communities to treatment or remediation efforts. For instance, the inventive methods hereof can be used to track the changes in the microbial community within the gut of a patient undergoing treatment for diseases such as Crone's or inflammatory bowel disease; such changes may be correlated with a positive or negative response to treatment, and would thus be highly useful both in terms of subsequent treatment decisions and prognosis. In alternate embodiments, the inventive methods and compositions hereof can be used to determine the composition of microbes inhabiting soil or other environments, or to characterize the composition in treatment facilities.

### EXAMPLES

### Example 1

### Serial Analysis of Ribosomal Sequence Tags in a Complex Microbial Community

DNA sample preparation. The microbial community genomic DNA was used in a previous study, which examined the prokaryotic diversity in different fractions of sheep rumen content. The DNA was sampled from the adhering fraction of a rumen digesta sample (Ad-H2) collected from a sheep fed a hay diet using the RBB+C method.

PCR amplification of the V1 region. According to the methods used herein, PCR primers were designed to target the V1 region of 16S rrs genes, which is the most variable region among the nine (V1-V9) hypervariable regions. The average length of the RST region derived from the V1 is 44.8 bp (ranging from 26 to 163 bp) among the 218 phylogenetic representative rrs genes in RDP. Theoretically, such a length can encode 3.66×10²⁴ (4^{44.8-4}) different RSTs, which are sufficient to accommodate all bacterial species in any ecosystem. However, based on an in silico analysis, RSTs provide somewhat lower resolution than longer rrs gene fragments. To increase resolution in some studies, the methods herein could be adapted to a longer hypervariable region. However, longer RSTs will mean lower throughput capacity.

Two universal primers were designed: BsgI-Bact64f (5′-dual biotin-TTT GAC CGT GCA GCY TAA YRC ATG CAA GTC G-3′) and BsgI-Bact109r1 (5′- dual biotin-TTT GAC CGT GCA GYY CAC GYG TTA CKC ACC CGT-3′). The bolded bases indicate extensions, which contain the recognition site (underlined bases) for the type IIS endonuclease BsgI. The BsgI-Bact64f differs from the universal primer Bac64f-BpmI used in SARST, with a different extension, longer primer length (18 bases), and reduced degeneracy (8, instead of 16). Except the extension, the BsgI-Bact109r1 is the same as the bacterial primer 109r1 of broad specificity described by Lane. The primers were synthesized and purified with HPLC by Integrated DNA Technologies (Coralville, Iowa).

Seven 50-μl PCR reactions (including one no-template control) were performed as previously described [Neufeld, 2004 #647], except for using BsgI-Bact64f and BsgI-Bact109r1 as primers and increased amount of DNA template (50 ng per reaction). The PCR products were pooled, and an aliquot of 0.5 μl was electrophoresed on an 8% (19:1) mini PAGE gel (all the PAGE gels used in iSARST were mini gels) at 100V/50 min on a Mini-PROTEIN II Cell (Bio-Rad Laboratories, Calif.) to visually check the PCR product. Following sampleion once with phenol/chloroform (P/C, pH 8.0), the PCR products were precipitated as described previously [Neufeld, 2004 #647], with the following modifications: 2.5 M ammonium acetate being replaced by 0.8 M LiCl, and no glycogen being added. After two washes with 75% ethanol, the DNA pellet was dried and then dissolved in 10 μl LoTE (3 mM Tris-HCl, 0.2 mM EDTA, pH 8.0).

Digestion with BsgI. The 10 μl purified PCR product was digested in a 20-μl reaction by 12 U BsgI (12 U/μl, New England BioLabs, Beverly, Mass.) at 37° C. for 3 hrs according to the manufacturer's protocol. Successful digestion was verified by electrophoresis of 0.5 μl on a mini 8% PAGE gel at 150V for 40 min.

Removal of primers with streptavidin beads. To the BsgI digest, 45.5 μl 2× magnetic bead binding buffer was added. Then RSTs were separated from the primers using Dynal M-280 beads (Dynal Biotech, Lake Success, N.Y.) and subsequently purified further by one P/C sampleion, as described previously. The RSTs were then precipitated as described above. The dried pellet was dissolved in 10 μl LoTE. The purified RSTs were visually checked by electrophoresis of 0.5 μl on an 8% mini PAGE gel as described above to ensure that undigested product was removed.

Ligation of RST toform concatemers. In a 0.5-ml tube, 5 μl purified RSTs was mixed with 2 μl 5× ligase buffer (Invitrogen Corp., Carlsbad, Calif.) and 2 μl water. Following gentle mixing, the tube was incubated at 40° C. for 2 min. After cooling on bench for 10 min, 2 μl T4 ligase HC (Invitrogen) was added. After gentle mixing and brief spinning, the ligation tube was incubated at 16° C. overnight. The whole ligation reaction was electrophoresed on a 1.5% agarose gel at 100V for 60 min. After staining with GelStar (BioWhittaker Molecular Applications, Rockland, Me.), the DNA was visualized under long UV. The fraction of 300-1,000 bp in length was excised out and sampled from the gel matrix using a MinElute Gel Sampleion Kit (QIAGEN Inc., Valencia, Calif.) according to the manufacturer's protocol (using 15 μl EB buffer).

Cloning, screening and sequencing of RST concatemers. The following were combined in a 0.5-ml tube: 4 μl 5×T4 DNA polymerase buffer, 14.7 μl purified RST concatemers, 0.8 μl dNTP (2.5 mM each), and 0.5 μl (1.2 U) T4 DNA polymerase (Invitrogen). The mixture was incubated at 12° C. for 15 min to blunt-end the DNA, and then at 75° C. for 20 min to inactivate the T4 DNA polymerase. The blunt-ended DNA concatemers were precipitated at −80° C. for 30 min, following addition of 80 μl water, 2 μl glycogen (20 mg/ml), 20 μl LiCl (4 M), and 300 μl ethanol. The DNA was pelleted by centrifugation at 4° C. for 15 min. The DNA pellet was washed once in 75% ethanol, dried on bench for 5 min, and finally dissolved in 7.5 μl water.

The vector pZErO™-2.1 (Invitrogen) was linearized in a 10 μl reaction containing 7 μl water, 1 μl 10× buffer REAct 2, 1 μl pZErO™-2.1, and 1 μl EcoRV (Invitrogen) at 37° C. for 30 min. The EcoRV was subsequently inactivated by incubation at 65° C. for 10 min. The blunt-ended RST concatemers was ligated into the linearized pZErO™-2.1 in a 10 μl ligation reaction consisting of the followings: 7.5 μl blunt-ended RST concatemers, 1 μl 10× T4 ligase buffer, 1 μl linearized pZErO™-2.1, and 0.5 μl T4 ligase HC (Invitrogen). The ligation reaction was incubated at 16° C. for 16 hrs. Two μl ligation product was directly electroporated into 50 μl electrocompetent E. coli TOP10 and rescued in 500 μl SOC medium. Aliquots of 50 and 100 μl transformation product were plated on LB plates containing kanamycin (50 μg/ml). Blue-white selection was achieved using X-gal to help indicate colonies carrying insert of appropriate size for screening and sequencing.

White colonies were inoculated into 96-well deep-well plates, with each well containing 0.5 ml LB supplemented with kanamycin (50 μg/ml). After overnight incubation at 37° C., 1 μl culture served the template in colony PCR screening using primers M13r and M13f(−20) as described previously. All white colonies screened were found to carry an insert. The above cultures were inoculated into the same type of plates containing fresh LB/Kan. Plasmid DNA was prepared from 24-hr cultures using a QIAprep® 96 Turbo Miniprep Kit (QIAGEN). The RST concatemers inserts were sequenced using the plasmid DNA at the DNA Genotyping/Sequencing Unit at The Ohio State University.

RST sequence analysis. Base-calling accuracy was visually confirmed and the vector portions at both ends were deleted using BioEdit (at URL www.mbio.ncsu.edu/BioEdit/bioedit.html). Then, individual RSTs were recognized by the head-tail border sequence 5′-ACGGGTCG-3′ (ACGGGT indicates the tail and the underlined bases indicate the head of two adjacent RSTs) using the Find function of BioEdit, and 4 “-” were manually inserted between adjacent RSTs (between the ACGGGT and CG) to separate them. In cases where the antisense strand was sequenced, the concatemers sequence was first converted to its sense strand using the Reverse-Complement function of BioEdit. Each edited sequence file was saved as a FASTA file from BioEdit. Individual RSTs were edited into FASTA format with a name indicating its physical position within each sequencing reaction box, with the box number serving the prefix. All the RSTs determined were combined into a single FASTA file, which served the input file of FastGroup (see below).

Unique RSTs were recognized by de-replication using FastGroup as described previously by [Neufeld, 2004, #647], but based on 97% sequence identity. Sequence identity to GenBank sequences was determined using BLAST. In cases where the most similar sequence is derived from uncultured bacteria, then the most similar sequence from a known bacterium was also recorded. Taxonomic assignment of individual RSTs were based on the phylogenetic affiliations of the most similar sequence(s) archived in RDP II version 9. Diversity indices were calculated as described previously. All grouped RSTs were deposited in the Gene Expression Omnibus database at URL www.ncbi.nlm.nih.gov/geo/. The annotated RSTs are also available at the Nature Biotechnology website.

Rarefaction analysis of RSTs was performed using the program aRarefactWin (available at URL www.uga.edu/˜strata/software/Software.html). The number of total RSTs (n) that need to be sequenced to achieve certain coverage was predicted by monomolecular curve analysis using the following equation: N=α(1−β.e[^{-κ.n}]), where N is the number of unique RSTs resulted from that coverage; the α (asymptote, equal to the maximum number of unique RSTs present in the library), β and κ values were calculated from the rarefaction curve using the SAS program (version 8) as described previously (Larue et al., 2004); and n represents the predicated number of total RSTs that need to be sequenced to achieve that coverage.

Results: Overview of iSARST FIG. 1 illustrates the iSARST procedure for generating RST concatemers. Two new primers, BsgI-Bact64f and BsgI-Bact109r1, are used to amplify the V1 region of the rrs gene but unlike SARST, BsgI digestion produces RSTs that are flanked with compatible 3′ overhangs (GT—for the sense strand and AC—for the antisense strand) which can be ligated directly in head-to-tail 4 orientation to form the desired RST concatemers. These new primers eliminated the need for the two biotinylated linkers used in SARST (Neufeld et al., 2004a, b) and thereby also eliminate all the steps required to ligate, digest, and remove the linkers. Overall, iSARST reduces the required materials and technical steps by more than half, reducing the RST clone library construction process to approximately three working days.

Analysis of the rumen microbiome by iSARST The iSARST procedure was used to produce a RST clone library from rumen microbiome DNA, and 768 E. coli colonies bearing a plasmid with an RST-containing insert were recovered and propagated in microtiter plates and stored at −80° C. From this library, 190 were randomly selected for plasmid DNA extraction and DNA sequencing. The sequence analysis showed these 190 clones contained 1,055 RSTs, and the numbers of RSTs per clone ranged from 1 to 19, with an average of 5.6 RSTs per clone. Based on a 95% sequence identity threshold, the 1,055 RSTs were further subdivided into 236 unique phylogenetic groups. (Accession No. GSM32172, at URL www.ncbi.nlm.nih.gov/geo/). The rarefaction and monomolecular curve analysis using this dataset estimates that the microbiome contains no more than 353 different phylotypes (based on [symbol] Ý95% sequence identity). The analyses also predict that 50% coverage of the bacterial diversity present in the sample requires 657 total RSTs, while 99% coverage requires 4,588 RSTs to be cloned and sequenced. Based on these measurements, the RSTs recovered from the 190 sequenced clones provide 67% coverage of the bacterial diversity present in the digesta sample. Furthermore, assuming the “average” clone contains 5.6 RSTs, the 768 E. coli colonies recovered will contain 4,224 RSTs, providing nearly 99% coverage of the bacterial diversity in the digesta sample, making this study one of the most comprehensive examinations of microbial diversity ever achieved in a single study of this type of samples.

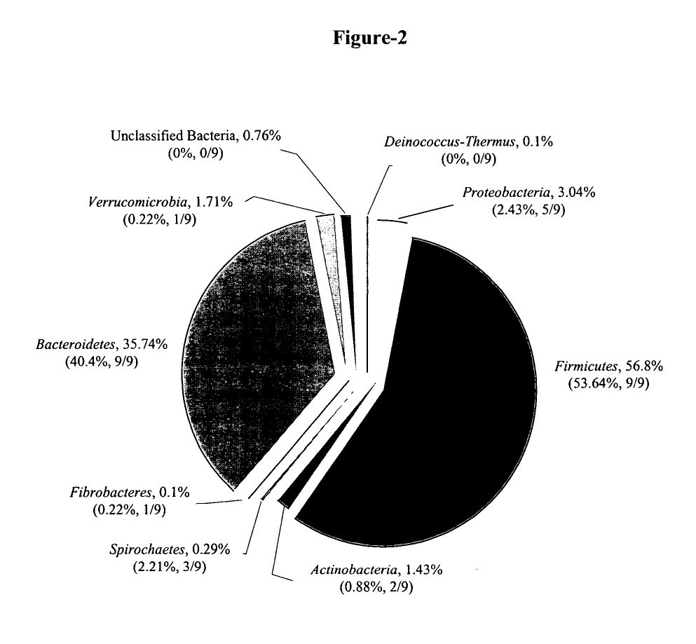

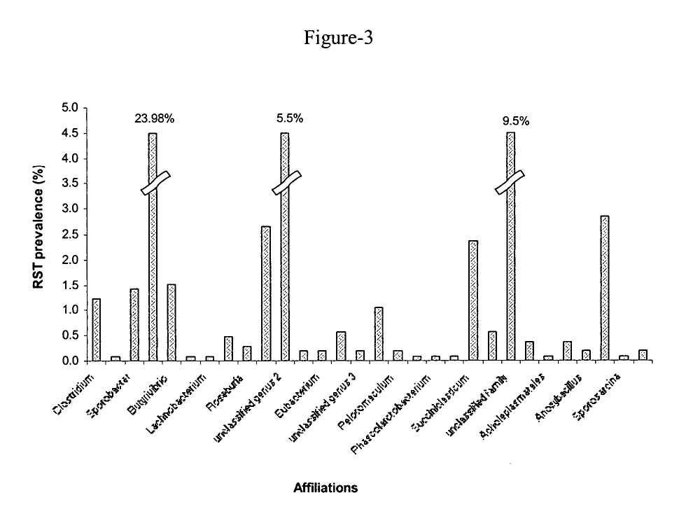

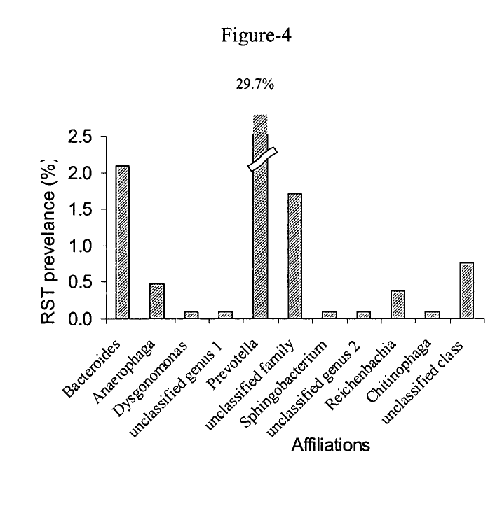

The 236 different RSTs identified in the rumen microbiome were assigned to 8 different Bacteria phyla (FIG. 2). These RSTs match database sequences with sequence identity ranging from 45% to 100% (at URL www.ag.ohio-state.edu/˜ansci/MAPLE/iSARST.htm). Most of the RSTs were affiliated with either Firmicutes (56.5% of the total) or Bacteroidetes (35.5% of the total), which is a characteristic typical of the microbiomes present in the digestive tracts of herbivores and humans. The RSTs affiliated with Firmicutes were further assigned to 27 genera as well as 2 unclassified orders (FIG. 3). Similar to the results obtained by ribosomal intergenic spacer analyses (RISA) with the same community DNA sample (Larue et al., 2004), RSTs representing an unclassified genus of Clostridiaceae and an unclassified genus within Lachnospiraceae were the most abundant RSTs (24% and 5.5% of the total, respectively). RSTs affiliated with an unclassified family of Clostridiales were also abundant in the clone library. Among the genera identified by RSTs for which culturable isolates are available, Ruminococcus, Succiniclasticum, and Paenibacillus appeared more abundant than the rest. Other relatively abundant RSTs appeared to be affiliated with species in the Clostridium (true Clostridium), Sporobacter, Butyrivibrio, and Desulfotomaculum genera. Among the RSTs identified as Bacteroidetes, Prevotella and Bacteroides were the most abundant, while some RSTs appeared related to an unclassified family and an unclassified class (FIG. 4). In total, nine genera of Bacteroidetes were identified besides one unclassified class and one unclassified family.

The Proteobacteria-like RSTs fell into all the five classes as well as an unclassified class within Proteobacteria (RDP II, Release 9.0). Almost 50% of these RSTs were affiliated with the Alpha Proteobacteria, and although most of the RSTs best matched sequences obtained from uncultured members of this class; RSTs were also identified that closely matched the rrs gene of Gluconacetobacter, Methylobacterium, Pseudomonas, Desulfomicrobium, or Campylobacter. Another five RSTs appeared to represent bacteria closely associated with Desulfovibrio spp, and the remaining RSTs fell into unclassified genera or families of Proteobacteria. The rest of the RSTs identified in the dataset were affiliated with Actinobacteria, Spirochaetes, Fibrobacter, Verrucomicrobia, Deinococcus-Thermus, and several unclassified genera.

DISCUSSION Based on >/=95% sequence identity, which is the lowest threshold typically used to demarcate operational taxonomic units (OTUs) with rrs genes (Hughes et al., 2001), the 1,055 RSTs sequenced in this study could be further divided into 236 phylotypes. The same community DNA sample has previously been analyzed by RISA (Larue et al., 2004) and in that earlier study only 50 phylotypes were identified by ribosomal intergenic spacer restriction fragment length polymorphism (RIS-RFLP) analysis of 96 randomly selected clones. The rarefaction and monomolecular curve analyses of the RISA dataset predicted a maximum of 86 phylotypes present in the microbiome, whereas the same analyses of the iSARST dataset predict a maximum of 353 phylotypes in the microbiome. As such, the iSARST procedure appeared to be an excellent way of assessing microbial diversity in this sample, compared to RISA-RFLP. To further evaluate the utility of iSARST to characterize microbial diversity, we collated the results produced from nine conventional rrs clone libraries that have been produced previously from 7 rumen samples of domesticated herbivores in four separate studies (Whitford et al., 1998; Tajima et al., 1999; Tajima et al., 2000; Koike et al., 2003). This composite dataset is comprised of 457 sequenced clones, which are assigned to 8 of the bacterial phyla identified by iSARST in this study. However, no single rrs clone library from rumen microbiomes contains the same breadth of coverage as the iSARST library, with the conventional rrs gene libraries recovering as few as 2 and no more than 5 of the phyla identified by iSARST. Within the most commonly identified phyla in gut microbiomes, Firmicutes and Bacteroidetes, more genera were identified by iSARST than in any other conventional rrs clone libraries (FIG. 3-4). Additionally, our RST clone library contains RSTs affiliated with all five classes of Proteobacteria, as well as Fibrobacter and Spirochaetes, all of which have long been recognized by cultivation-based and microscopic studies as residents of rumen microbiomes, but are rarely recovered in rrs clone libraries (Whitford et al., 1998; Tajima et al., 1999; Larue et al., 2004). In addition to genera frequently represented in other rrs clone libraries of rumen microbiomes, some RSTs were identified that are affiliated with genera that have been reported present in the gastrointestinal tracts of other animals. These include Sporobacter and Paenibacillus from termites (Grech-Mora et al., 1996; Wenzel et al., 2002), Sphingobacterium from ants (Jaffe et al., 2001), and Acholeplasma from midge (Campbell et al., 2004); as well as Anoxybacillus from manures (Pikuta et al., 2000). Furthermore, iSARST produced RSTs that are not closely affiliated with existing phylogenetic lineages. For instance, there were a large number of RSTs only distantly related with existing lineages of Clostridiaceae, in accordance with our previously published findings with the same digesta sample (Larue et al., 2004), as well as the findings of Nelson et al. (Nelson et al., 2003) from their studies of non-domesticated ruminants. These direct and indirect comparisons show that iSARST not only effectively produces a comprehensive representation of the bacterial diversity known to be numerically predominant in this microbiome, but also includes RSTs representing bacterial groups that have only been rarely recovered in conventional rrs gene libraries, as well as novel, not yet cultured bacterial groupings.

Both serial analysis of gene expression (SAGE) and SARST employ the same strategy: the creation of sequence tag concatemers to improve sequencing efficiency. However, the RSTs must be ligated in a head-to-tail orientation to prevent the formation of stem-loop structures between adjacent homologous RSTs that interfere with DNA sequencing (Neufeld et al., 2004a). In SARST, head-to-tail ligation of RSTs was ensured by using two biotinylated linkers, and this necessitated the inclusion of several lengthy and technically cumbersome steps (Neufeld et al., 2004a, b). In iSARST, the BsgI recognition site in each primer extension was positioned in such a distance that a single BsgI digestion of the PCR product is all that is needed to generate RSTs with cohesive overhangs that ensure a head-to-tail orientation of the RSTs (FIG. 1). Compared to the original method, iSARST reduces the time and the costs associated with RST library construction by more than 50%. Additionally, the elimination of BpmI digestion also eliminates the probability to cut the RSTs by this type IIS restriction enzyme. Our approach to primer design for iSARST can also be used to design primers to generate RSTs from other hypervariable regions of rrs genes, and the recently reported SARST-V6 employs a similar approach. However, unlike SARST-V6, which employs TA cloning following addition of A overhangs to the blunt-ended concatemers (Kysela et al., 2005), iSARST employs blunt-end ligation, which is shown to improve the cloning efficiency of SAGE concatemers (Koehl et al., 2003). Further, since it is the most hypervariable region located near the 5′ end of rrs genes, V1-9 RSTs permit the recovery of nearly complete rrs gene sequence and the design of more specific primers or probes.

Similar to SARST (Neufeld et al., 2004a) and SARST-V6 (Kysela et al., 2005), the average number of RSTs per clone was about 6, though some of our clones contained as many as 19 RSTs. As such, there is the potential to increase the number of RSTs that can be accurately sequenced within the average read lengths achieved by most DNA sequencing facilities. In that context, methods developed and used to increase concatemer length for SAGE, such as the incubation of the concatemers at 65° C. for 15 min prior. to gel sizing (Kenzelmann and Mahlemann, 1999), should be equally applicable with iSARST. Even without these modifications, the number of iSARST clones to be sequenced for extensive coverage of a complex microbiome is well within the capacity of most DNA sequencing facilities, and the budgets of many research laboratories (the reagent costs associated with the construction and analysis of this library was less than $2,500).

While the RSTs are expected to provide a lower phylogenetic resolution than longer rrs fragments or full-length gene sequences, as the most variable region within rrs genes (Yu and Morrison, 2004), V1-RSTs could be resolved with confidence to genus, and in many cases, to the species level of identification (Neufeld et al., 2004a). To further evaluate the phylogenetic resolution of the V1-RSTs, we also analyzed the rrs sequences for the genus Escherichia deposited in RDP II. The average sequence identity among the 96 nearly full-length Escherichia rrs sequences is 97.5%, while their V1-RSTs are only 92.3% identical. When the comparison is further narrowed to E. coli strains, the identity of the full-length gene is 97.8%, but only 94.3% 10 when the V1-RSTs alone are considered. An in silico analysis of the V1-RSTs of the 218 phylogenetic representative rrs sequences listed in RDP II also showed there are only 5 instances (from a total of 23,653 pairwise comparisons) where an identical RST would be produced from two different bacteria. Collectively, these observations support the contention that the V1-RSTs can provide a suitable degree of resolution, even among closely related species; and the incorrect phylogenetic assignment of an RST should not only be rare, but not significantly undermine the validity of iSARST analyses.

Widely used methods in microbial ecology research, such as DGGE, TGGE, T-RFLP and ARISA all need to be combined with fragment recovery, PCR, cloning, and sequencing to provide speciation, which requires additional investments in time and money. Microarrays are now being developed for well characterized microbiomes to support high-throughput analyses of select microbial populations (Koizumi et al., 2002; Loy et al., 2002; Chandler et al., 2003), but they require pre-determined rrs sequences for probe design. In that context, iSARST provides sequence-based information that is useful for the recovery of full length rrs clones, as demonstrated previously (Neufeld et al., 2004a) and that can also be used to help design primers for real-time PCR assays to quantify particular bacteria. For these reasons, we consider iSARST to be an informative and technically amenable method for many microbial ecologists currently using DGGE and related methods to examine bacterial diversity in any microbiome. These results not only help validate iSARST and related methods as a useful tool in microbial ecology studies, but also provides further evidence to support the tenet that the biofilms adherent to plant biomass in herbivores are genetically more diverse than previously perceived.

Some of the techniques that are known in the art are described in the following references, which are incorporated herein in their entirety: Neufeld, J. D. Z. Yu, W. Lam, W. W. Mohn (2004). Serial analysis of ribosomal sequence tags (SARST): a high-throughput method for profiling complex microbial communities. Environmental Microbiology 6:131-144. Velculescu, V. E., Zhang, L., Vogelstein, B., and Kinzler, K. W. (1995). Serial Analysis Of Gene Expression. Science 270, 484-487.

1. A method for genetic analysis in complex microbial communities, comprising the steps of: amplifying a sample containing polynucleotides isolated from a microbial community to provide amplified polynucleotide products using one or more primer pairs, each of said primer pairs having sequences that are complementary to a targeted sequence, wherein each of the primers comprise extensions having restriction endonuclease recognition sites, which, upon digestion of the amplified polynucleotide products with corresponding restriction endonuclease reagents, provide polynucleotide ribosomal sequence tags comprising overhangs that are complementary at their 3′ and 5′ ends; isolating the amplified polynucleotide products; digesting the amplified polynucleotide products with the corresponding restriction endonuclease reagents to provide polynucleotide ribosomal sequence tags; separating the tags from the primers; concatenating the tags in a head to tail orientation.

2. The method according to claim 1 wherein the concatenated tags are subjected to sequence analysis.

3. The method according to claim 1 wherein the primers are complementarity to the V1 region of 16S rrs gene.

4. The method according to claim 3 wherein the restriction endonuclease recognition sites of the primers are BsgI restriction endonuclease sites.

5. The method according to claim 4, wherein the primers each have a dual biotin label.

6. The method according to claim 5, wherein the primers comprise a first primer having the sequence 5′-TTT GAC CGT GCA GCY TAA YRC ATG CAA GTC G-3′ (SEQ ID NO: 1) and a second primer having the sequence 5′-TTT GAC CGT GCA GYY CAC GYG TTA CKC ACC CGT-3′ (SEQ ID NO: 2).

7. A method for genetic analysis of complex microbial communities, comprising the steps of: amplifying a DNA sample from a microbial community to provide amplified polynucleotide products using primer pairs having sequences that are complementary to one of the V1 through V9 regions of the ribosomal genes, wherein each of the primers comprise extensions having restriction endonuclease recognition sites, which, upon digestion of the amplified polynucleotide products with corresponding restriction endonuclease reagents, provide polynucleotide ribosomal sequence tags comprising overhangs that are complementary at their 3′ and 5′ ends; isolating the amplified polynucleotide products; digesting the amplified polynucleotide products with the corresponding restriction endonuclease reagents to provide polynucleotide ribosomal sequence tags; separating the tags from the primers; concatenating the tags in a head to tail orientation.

8. The method according to claim 7 wherein the concatenated tags are subjected to sequence analysis.

9. The method according to claim 7 wherein the restriction endonuclease recognition sites of the primers are BsgI restriction endonuclease sites.

10. The method according to claim 9, wherein the primers each have a dual biotin label.

11. The method according to claim 10, wherein the primers are complementarity to the V1 region of 16S rrs gene and comprise a first primer having the sequence 5′-TTT GAC CGT GCA GCY TAA YRC ATG CAA GTC G-3′ (SEQ ID NO: 1) and a second primer having the sequence 5′-TTT GAC CGT GCA GYY CAC GYG TTA CKC ACC CGT-3′ (SEQ ID NO: 2).

12. A method for genetic analysis of complex microbial communities, comprising the steps of: amplifying a DNA sample from a microbial community to provide amplified polynucleotide products using primer pairs having sequences that are complementary to one or more antibiotic or antimicrobial resistance genes, wherein each of the primers comprise extensions having restriction endonuclease recognition sites, which, upon digestion of the amplified polynucleotide products with corresponding restriction endonuclease reagents, provide polynucleotide ribosomal sequence tags comprising overhangs that are complementary at their 3′ and 5′ ends; isolating the amplified polynucleotide products; digesting the amplified polynucleotide products with the corresponding restriction endonuclease reagents to provide polynucleotide ribosomal sequence tags; separating the tags from the primers; concatenating the tags in a head to tail orientation.

13. The method according to claim 12 wherein the concatenated tags are subjected to sequence analysis.

14. The method according to claim 12 wherein the restriction endonuclease recognition sites of the primers are BsgI restriction endonuclease sites.

15. The method according to claim 14, wherein the primers each have a dual biotin label.

16. Isolated polynucleotide primers for amplifying and isolating DNA tags located within a targeted genetic region from one or more microbial organisms, comprising polynucleotides having sequences that are complementary to sequences within the targeted genetic region and extensions designed to enable direct concatenation of two or more isolated tags in a head to tail orientation without the need for intermediate linkers.

17. Isolated polynucleotide primers according to claim 16, wherein the primers comprise a first and a second primer, each primer comprising a dual biotin label and a BsgI restriction endonuclease site, said first primer having the sequence 5′-TTT GAC CGT GCA GCY TAA YRC ATG CAA GTC G-3′ (SEQ ID NO: 1), and said second primer having the sequence 5′-TTT GAC CGT GCA GYY CAC GYG TTA CKC ACC CGT-3′ (SEQ ID NO: 2).

18. A kit for evaluating microbial populations, comprising one or more primer sets used to provide amplified polynucleotide products in appropriate containers, each of said one or more primer sets comprising primer pairs for targeting specific genetic regions in a microbial genome, each of said primers having extension sequences encoding for restriction endonuclease recognition sites which, upon digestion of the to provide amplified polynucleotide products with corresponding restriction endonuclease reagents, produce isolated polynucleotide tags from within the targeted region of DNA, said tags comprising overhangs that are complementary at their 3′ and 5′ ends; reaction components in appropriate containers comprising restriction endonucleases corresponding to and specific for the restriction endonuclease recognition sites on the one or more primer sets; at least one ligase for producing concatemers, such as T4 ligase, in an appropriate container; DNA polymerase, such as T4 DNA polymerase, in an appropriate container; and a cloning vector in an appropriate container.

19. A kit according to claim 18 wherein the primers have complementarity to one of the V1 through V9 regions of the ribosomal genes or to one or more antibiotic or antimicrobial resistance genes, or combinations thereof.

20. A kit according to claim 19, comprising a first and a second primer, each primer having complementarity to the V1 region of 16S rrs gene, a BsgI restriction endonuclease recognition site, and a dual biotin label, the first primer having the sequence 5′-TTT GAC CGT GCA GCY TAA YRC ATG CAA GTC G-3′ (SEQ ID NO: 1), and the second primer having the sequence 5′-TTT GAC CGT GCA GYY CAC GYG TTA CKC ACC CGT-3′ (SEQ ID NO: 2).
